(19) United States
(12) Patent Application Publication
(10) Pub. No.: US 2015/0073385 A1
(43) Pub. Date: Mar. 12, 2015

(54) REMOTE RESERVOIR MICRONEEDLE DRUG DELIVERY SYSTEMS

(71) Applicants: Bradley Lyon, Arcadia, CA (US); Adrianus I. Aria, Cambridge (GB); Morteza Gharib, Altadena, CA (US)

(72) Inventors: Bradley Lyon, Arcadia, CA (US); Adrianus I. Aria, Cambridge (GB); Morteza Gharib, Altadena, CA (US)

(73) Assignee: CALIFORNIA INSTITUTE OF TECHNOLOGY, Pasadena, CA (US)

(21) Appl. No.: 14/481,889

(22) Filed: Sep. 9, 2014

Related U.S. Application Data
(60) Provisional application No. 61/876,072, filed on Sep. 10, 2013.

(51) Int. Cl. A61M 37/00

(52) U.S. Cl. CPC A61M 37/0015; A61M 2037/0023; A61M 2037/0061; A61M 2037/003
USPC 604/506; 604/173

(57) ABSTRACT

Transdermal drug delivery patch devices are described in which microneedle array insertion or application forces are decoupled or separated from drug delivery actuation or application forces. Dual-reservoir devices are described in detail for such purposes as are associated methods of use in various examples.

### RELATED APPLICATIONS

This filing claims the benefit of and priority to U.S. Provisional Patent Application Ser. No. 61/876,072 filed Sep. 10, 2013, which is incorporated by reference herein in its entirety for all purposes.

### FIELD

This application relates to transdermal drug delivery methods and devices incorporating micro-scale and/or nano-scale needles (collectively referred to as microneedles) therein.

### BACKGROUND

In the last few decades, drugs of increased potency have been developed in the pharmaceutical field. However, the administration of such drugs has been limited due to poor absorption, enzymatic degradation in the gastrointestinal tract, and/or painful delivery using intravascular injection.

The potential for a solution to such problems utilizing transdermal drug delivery patches has been stymied thus far by limited skin permeability. Recently, however, advances in the manufacture of hollow microneedles and arrays thereof have enabled transdermal patch delivery of formulated liquid drugs otherwise used in hypodermic injection. Indeed, designs have been delineated for skin patches that enable a so-called “push-to-deliver” type architectures for transdermal microneedle drug delivery. See, e.g., US Patent Publication No. 2010/0196446.

The push-to-deliver architecture is beneficial in that the drug delivery patch may have the familiar look and feel of a band-aid. The drug is released into the skin by simply pushing on the device, which eliminates the need for the pumps used in other transdermal drug delivery device designs. In typical push-to-delivery skin patches, the patient presses down on a thin deformable membrane in a single motion that simultaneously presses microneedles into the skin and releases the drug from a reservoir in fluid communication with those microneedles. By eliminating the pumps, there is no need to provide power to the patch to operate the pumps, and for this and other reasons the overall cost and size of a transdermal patch can be reduced.

The subject matter described herein contains improvements to existing push-to-deliver devices.

### SUMMARY

Push-to-deliver patch designs can be cumbersome and difficult to design and operate given that the simultaneous action of needle insertion and drug delivery creates a high risk of drug being released prior to the tips of the microneedles being sufficiently inserted into the skin. A poorly timed relationship between microneedle insertion and drug delivery can result in drug loss and dosing errors. Devices, systems, and methods are described herein that can reduce these risks.

Multiple example embodiments are set forth of dual reservoir remote-reservoir drug delivery devices. In many of these embodiments, the force applied to insert microneedles into the skin of a user or patient is decoupled from the force applied to actuate drug flow. This allows the microneedles to be properly set in the skin before actuating release of drug.

Each of the embodiments described herein can employ a composite carbon nanotube (CNT) microneedle array as described in U.S. patent application Ser. No. 14/231,267 (CIT-6508), which is incorporated by reference herein in its entirety and for all purposes. Other types of microneedles, including those that utilize a lumen within each individual nanotube, can be employed as well. Overall, microneedle drug delivery systems, devices, and methods are described as an optionally self-administered, painless alternative to standard hypodermic injection.

Also provided herein are example embodiments of methods of manufacture and methods of use of the embodiments of all of the devices and systems described herein. The methods of use may include methods of drug delivery, of inoculation or vaccination, of analyte acquisition and analysis, and the like.

Still other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments in this or any section hereof be construed as limiting the appended claims, absent express recitation of those features therein.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. However, the subject of photographs and related drawing(s) may be taken and used for antecedent basis in terms of scale.

FIG. 1A is a cross-sectional schematic of a prior art single reservoir transdermal skin patch.

FIG. 1B is a cross-sectional schematic depicting an example embodiment of a dual reservoir transdermal skin patch device.

FIG. 2 is an assembly view depicting an example embodiment of a transdermal skin patch device.

FIG. 3 is a flowchart depicting an example embodiment of a method of using an example embodiment of a transdermal skin patch device.

FIGS. 4A-4C are photographs of an example embodiment of a transdermal skin patch device.

FIG. 5 is a photograph of an example of puncture marks and dye delivery by an example embodiment of a transdermal skin patch device.

### DETAILED DESCRIPTION

The present subject matter is not limited to the particular embodiments described, as those are only examples and may, of course, vary. Likewise, the terminology used herein is for the purpose of describing particular embodiments only, and is not intended to be limiting, since the scope of the present disclosure will be limited only by the appended claims.

Embodiments of the reservoir devices described herein separate the action of microneedle penetration of the skin from actuation of drug flow. In many embodiments this can be done by moving the majority of drug reservoir content from directly over the microneedles, as in previous designs, to an offset or remote position (e.g., in terms of location of a Primary Reservoir or Drug Reservoir I as described below). A minimal volume reservoir (e.g., a Secondary Reservoir or Drug Reservoir II as described below) can be maintained directly over or facing the microneedles to provide steady flow of the drug to the microneedles during delivery. Additionally, a minimal volume channel can optionally connect Drug Reservoir I to Drug Reservoir II.

With any given patch, properly placing the microneedles into a user's skin typically requires the application of a small but steady force directly over the microneedles on the order of 1 Newton (N) per needle. When inserting the microneedles, it is desirable to remain cognizant of the qualitative force applied as well as to achieve proper contact with taut skin occurs. For some users or patients (especially for new patients or patients with poor coordination), it may take several moments to properly place the microneedle before drug delivery should be commenced. With the offset position of the drug reservoir relative to the microneedles, the patient directly applies force to the skin patch rather than the liquid reservoir. Such an approach can be advantageous in minimizing accidental drug solution discharge wherein any such discharge is advantageously less than about 1 microliter (μL) of fluid driven through the microneedles when inserting the microneedles into skin.

Several other criteria may be considered, as well, to minimize accidental drug discharge. For example, the volume of Drug Reservoir II may be minimized to reduce the total amount of drug that can be potentially released. For example, the volume may be between 5 μL and 30 μL to limit potential drug loss when pressing the microneedles into the skin. In a related approach, a volume ratio of the Primary Reservoir to the Secondary Reservoir may be such that the volume of the Primary Reservoir is at least double that of the Secondary Reservoir.

Also, the thickness of the skin patch and/or the elastic modulus of its material may be set such that the skin patch material cannot be significantly deformed in the force range of several Newtons, advantageously up to at least 30 N of compressive force. For soft materials, like polydimethylsiloxane (PDMS), the thickness criteria can be more important. For hard materials, like an acetal plastic, the elastic modulus can be more important than thickness. In the latter case, the elastic modulus may be at least about 1 megapascal (MPa).

Likewise, the elastic modulus of a top membrane material covering the Drug Reservoir I may be significantly lower than that of a skin patch material such that the delivery force applied onto Drug Reservoir I is localized only to Drug Reservoir I and not transferred to the microneedles or other parts of the skin patch. Further, the overall inlet resistance to Drug Reservoir II may be set smaller or lower than the outlet resistance of the microneedles such that any shape deformations to Drug Reservoir II are most likely to drive flow into Drug Reservoir I rather than through the microneedles. Still other features and/or advantages associated with the above are possible as well.

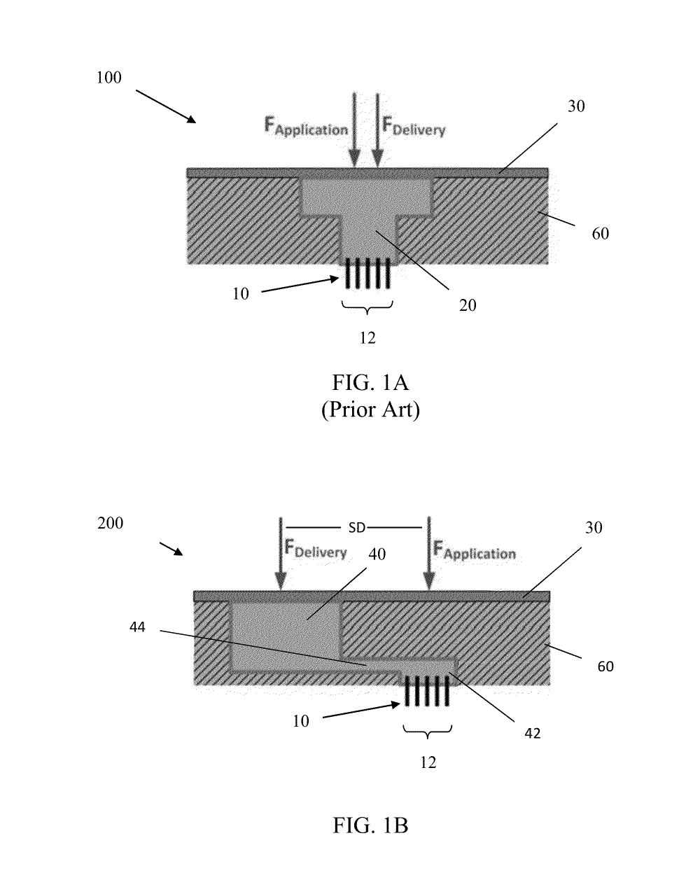

Several of the aspects above may be further appreciated in relation to devices 100 and 200 illustrated in FIGS. 1A and 1B, respectively. In each figure, the application force arrow (F_{Application} or F_{A}) denotes optimum position on each transdermal drug delivery patch 100, 200 to apply force to achieve proper placement of each microneedle 10 in the skin. The delivery force arrow (F_{Delivery} or F_{D}) denotes optimum position on each patch to apply force to actuate drug flow.

With the prior art single-reservoir patch 100, microneedle application and delivery actuation occur simultaneously where F_{A} and F_{D} are coincident or along the same or directly adjacent line of action (wherein the line separation shown in FIG. 1A is exaggerated for illustration purposes only). As such, drug loss and dosing error are prone to occur by virtue of compression of reservoir 20 during needle insertion. With example embodiments of the remote or dual reservoir patch 200, microneedle application can be achieved independently of delivery actuation by virtue of the separation distance (SD) between F_{A} and F_{D} to properly actuate the device.

In some example embodiments, the lines of action (F_{A} and F_{D}) may be separated by about at least an average user finger's width (e.g., 1 centimeter (cm)) or more. In one example embodiment, the gap or distance SD between the lines of action (F_{A} and F_{D}) is between about 1 cm and about 5 cm.

Whereas device 100 includes a single reservoir 20 (in this case shown with a variable cross section accommodating a push-pad membrane 30 that can provide actuation in an upper region), two discrete reservoirs 40, 42 are shown in device 200. These are in fluid communication though a tube or channel 44. Yet, reservoir 40 (alternatively referred to as Reservoir I or the Primary Reservoir) is physically separated by channel 44 to be remote or separated from reservoir 42 (alternatively referred to as Reservoir II or the Secondary Reservoir). In some embodiments, channel 44 may range from about 3 millimeters (mm) to about 10 mm in length. In shorter configurations, channel 44 could be referred to as an orifice.

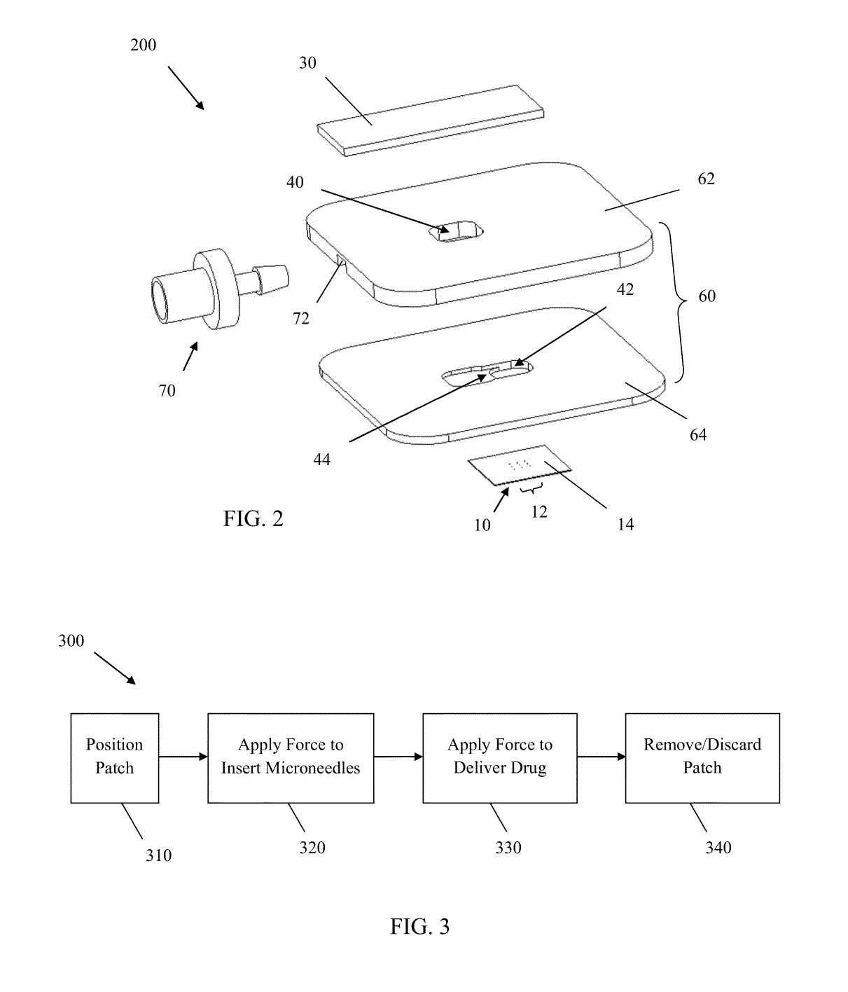

In either device 200, reservoirs 40 and 42 and channel 44 and related features may be formed in a base 60 comprising flexible silicone (or other) material. The aforementioned push pad membrane 30 may be formed by a relatively thin flexible sheet comprising silicone or other material covering some or all of base 60. An array 12 of the microneedles may be secured directly within base 60, using a backing 14 (e.g., as further described in connection with FIG. 2) or otherwise as may be appreciated by those with skill in the art.

FIG. 2 illustrates another example embodiment of device 200. Here, base 60 is formed by a pair of block pieces 62, 64. Upper base piece 62 has the Primary Reservoir 40 (Reservoir I) formed therein. Lower base piece 64 has the Secondary Reservoir 42 (Reservoir II) and connecting channel 44 formed therein. When assembled with array base 14 and cover membrane 30, a closed system may be formed. Such assembly may be accomplished using plasma bonding, with ECOFLEX or PDMS adhesive between the respective layers or otherwise.

A fill fitting (e.g., syringe adapter 70) may be received within a channel 72 integrated in base members 62, 64 in fluid communication with the reservoir(s) for filling or priming the patch. The filling may be through injection or by applying vacuum to draw drug solution into the reservoirs through the microneedles when submerged in such fluid. The latter approach may be useful in avoiding the possibility of air bubbles at the interface between the secondary reservoir and the microneedles which can cause dosing errors during delivery. Likewise, a vacuum introduction approach can also be used to fully eliminate a syringe adapter by piercing the base with a sharp object such as a small hypodermic needle. In which case, after filling the drug reservoirs, the hole left by the needle may be injected with a thermosetting resin, such as PDMS, to allow the reservoir to be sealed and the hypodermic needle to be removed.

However prepared and/or ultimately packaged, flowchart 300 in FIG. 3 details an example embodiment of the use of a patch device 200. Specifically, at 310 a user (or a person assisting the user) positions patch device 200 against the skin where drug is to be delivered. At 320, force is applied (typically with a finger) to insert the microneedles 10 into the skin. Then, in a separate action or motion, force is applied to deliver the drug solution contained within the device at 330. As described above, such drug will be in solution contained (primarily) in reservoir 40. The process may be considered complete upon drug delivery. Typically, however, the patch will be removed and discarded as shown at 340.

### EXAMPLE

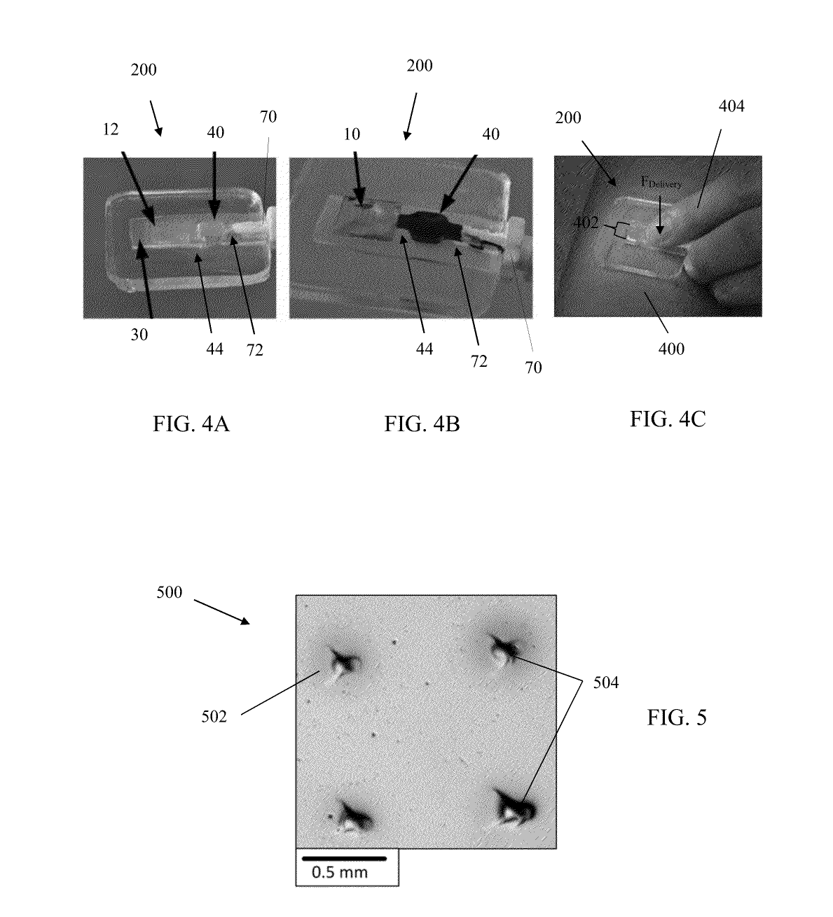

An example embodiment of the subject dual reservoir skin patch 200 including CNT-polyimide microneedles 10 fabricated according to FIG. 2 is shown in FIG. 4A (top side) and FIG. 4B (bottom or under side). Use of the device is pictured in FIG. 4C with device 200 set or positioned on a user's skin 400 at a treatment site 402 with membrane 30 (and hence reservoir 40) compressed by a user's finger 404 following microneedle array insertion.

In this example patch 200 was made from PDMS (Polydimethylsiloxane, Sylgard 184 silicone elastomer) and deformable membrane 30 made from a commercial silicone (Ecoflex 30) that deforms more easily than the PDMS. Patch 200 is large enough to be easily handled by the user or patient (i.e., 3.6 cm×3.6 cm) while allowing for targeted release in a 2 mm×2 mm area by nine microneedles 10 in the array 12.

For illustrative purposes, patch 200 was loaded with methylene blue dye in water. The patch held approximately 114 μL of fluid with 84 μL held in reservoir 40 (Drug Reservoir I), 22 μL held in reservoir 42 (Drug Reservoir II) and 8 μL held in the channel 44 connecting the two reservoirs. (Note reservoir 40 and channel 44 are shown unfilled in FIG. 4A and as-filled in FIG. 4B.) An inlet 72 (shown with syringe adapter 70 inserted therein) to the patch immediately upstream of reservoir 40 was used for drug reservoir loading from a syringe for the ease of conducting experiments.

In one experiment, water was delivered into air via nine CNT-polyimide microneedles. Hand (i.e., finger-based) actuation of reservoir 40 (by depressing membrane 30 using an index finger 404 as shown—in this respect—in FIG. 4C) yielded an estimated average flow rate of 339 μL/minute demonstrating efficient liquid delivery.

FIG. 5 pictures the results of another experiment showing penetration into agarose gel 500 with an example embodiment of the dual reservoir skin patch. A water-methylene blue dye mixture 502 was delivered into the agarose gel to demonstrate that the patch was free of any inadvertent, unintended or accidental liquid release during microneedle application. Since methylene blue solution is readily absorbed into the gel, any such leakage before microneedle insertion would be visible on the gel surface. When used as described in connection with FIG. 3, results with a device 200 employing a 2×2 (i.e., 4 needle) microneedle array 12 show four distinct punctures 504 into the gel with dye diffusing only from the punctures without any trace of dye leakage or spillage on the surface.

Variations

All of the following references are incorporated by reference herein in their entirety and for all purposes: U.S. Pat. No. 7,955,644; US Patent Application Publication Nos. 2010/0196446; 2011/0250376; 2012/0021164; 2012/0058170; and 2013/0178722; B. Lyon, A. I. Aria, et al, APS DFD Meeting, “Carbon Nanotube Micro-Needles for Rapid Transdermal Drug Delivery,” San Diego, Calif., Nov. 18, 2012; B. Lyon, A. I. Aria, M. Gharib, “Feasibility Study of Carbon Nanotube Microneedles for Rapid Transdermal Drug Delivery,” Mater. Res. Soc. Symp. Proc. Vol. 1569, DO1: 10.1557/op1.2013.803; B. Lyon, A. I. Aria, M. Gharib, “Carbon Nanotube-Polyimide Composite Microneedles for Rapid Transdermal Drug Delivery”, Society for Biomaterials Meeting, Boston, Mass., April 2013; and B. Lyon, A. I. Aria, M. Gharib, “Feasibility Study of Carbon Nanotube Microneedles for Rapid Transdermal Drug Delivery,” MRS Spring Meeting, San Francisco, Calif., April 2013. All of the teachings, techniques and other features described these references may be employed in all embodiments hereof.

It is contemplated that any feature of the embodiment variations described may be set forth and claimed independently or in combination with any one or more of the features described herein. Reference to a singular item includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Express recitation of every possible combination and substitution is overly burdensome, especially given that the permissibility of each and every such combination and substitution will be readily recognized by those of ordinary skill in the art upon reading this description.

Where a range of values is provided, it is noted that each intervening value, to the tenth of the unit of the lower limit unless the context clearly dictates otherwise, between the upper and lower limit of that range and any other stated or intervening value in that stated range, is encompassed within the disclosure and can be claimed as an sole value or as a smaller range. Where the stated range includes one or both of the limits, ranges excluding either or both of those included limits are also included in the disclosure.

Where a discrete value or range of values is provided, it is noted that that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should a claim be limited to a particular value or range of values absent explicit recitation of that value or range of values in the claims. Values and ranges of values are provided herein merely as examples.

The scope of the claims originally filed herewith is not intended to define the limits of the subject matter that may be claimed on the basis of this description. Broader and/or altogether different subject matter may, in fact, be claimed in the future without departing from the scope of the present description.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A medical device in the form of a transdermal drug delivery patch having a horizontal surface for placement upon a user's skin, the device comprising:
a base, the base including a first reservoir and a second reservoir, the first reservoir in fluid communication and horizontally offset from the second reservoir, and
a plurality of luminal microneedles in an array, the array secured to the base with the microneedles in direct fluid communication with the second reservoir,
wherein the horizontal offset between the first and second reservoirs is sufficient for pressing the microneedles into the skin without compressing the first reservoir.

2. The device of claim 1, wherein the first and second reservoirs are the only reservoirs in the device and they are adapted to be filled with liquid drug solution.

3. The device of claim 1, wherein the first and second reservoirs are integrally formed in the base.

4. The device of claim 1, further comprising a backing for the microneedle array, the backing securing the array to the base and covering the second reservoir.

5. The device of claim 1, wherein the first and second reservoirs are connected by a channel.

6. The device of claim 1, wherein the first reservoir is covered by a flexible membrane adapted to be depressed to reduce the volume of the first reservoir and force drug solution into the second reservoir and out the microneedles.

7. The device of claim 6, wherein the membrane is significantly more deformable than the base so that delivery force applied to the membrane is localized to the first reservoir.

8. The device of claim 1, wherein the second reservoir is adapted to avoid compression when pressing the microneedles into the skin.

9. The device of claim 8, wherein the compression is avoided up to at least 30 Newtons of compressive force.

10. The device of claim 8, wherein the adaptation to avoid second reservoir compression is provided by the base having an elastic modulus of at least about 1 MPa.

11. The device of claim 8, wherein the adaptation to avoid second reservoir compression is provided by base thickness.

12. The device of claim 1, wherein a volume of the first reservoir is at least double a volume of the second reservoir.

13. The device of claim 1, wherein the second reservoir is sized to between 5 μL and 30 μL to limit potential drug loss when pressing the microneedles into the skin.

14. The device of claim 1, where an inlet resistance of the second reservoir is lower than an outlet resistance of the microneedles whereby any shape deformation to the second reservoir drives flow into the first reservoir preferentially instead of through the microneedles.

15. A method of transdermal drug delivery for a user, the method comprising:
placing a drug delivery patch on skin of the user;
first pressing the patch in one location to insert microneedles from the patch into the skin; and
second pressing the patch in another, offset location to drive fluid from the patch through the microneedles.

16. The method of claim 15, wherein the first pressing drives no substantial amount of fluid through the microneedles.

17. The method of claim 16, wherein no amount of fluid is driven through the microneedle by the first pressing.

18. The method of claim 16, wherein less than about 1 μL of fluid is driven through the microneedles by the first pressing.

19. The method of claim 16, wherein the second pressing actuates a reservoir remote from the microneedles.

20. The method of claim 16, wherein the second pressing is aligned with a first reservoir in fluid communication with a second reservoir that is aligned with the first pressing.
